(19) United States
(12) Patent Application Publication
(10) Pub. No.: US 2009/0060942 A1
(43) Pub. Date: Mar. 5, 2009

(54) Detoxification Composition and Method of Detoxifying the Body

(76) Inventors: Ann Marie O'Mara, Limerick (IE); Brendan Joseph O'Mara, Clare (IE)

Correspondence Address: AMIN HALLIHAN, LLC, 444 NORTH ORLEANS STREET, SUITE 400, CHICAGO, IL, 60654, US

(21) Appl. No.: 11/848,264

(22) Filed: Aug. 31, 2007

(51) Int. Cl. A61K 36/05; A61P 39/00; A61P 9/00

(52) U.S. Cl. 424/195.17

(57) ABSTRACT

The present invention provides a detoxification composition including alginate, and ulva rigida extract. The present invention further provides a method of detoxifying and cleansing the human body of mutagens, and toxins; reducing cholesterol levels; and preventing or protecting against colon cancer.

### FIELD OF THE INVENTION

The present invention relates to a composition containing alginate used for detoxifying and cleansing a body, particularly a human body, of toxins and mutagens. The present invention further relates to a composition containing alginate used to prevent and protect against colon cancer and reduce cholesterol levels in humans and other animals. The present composition is designed to be ingested and taken as a beverage.

### BACKGROUND OF THE INVENTION

The known health benefits of dietary fiber include lowering the risk of colon cancer, heart disease, lowering serum lipids, and improving glucose tolerance due to the high water-holding capacity, viscosity enhancing effect, gel forming properties, high adsorption capacity including cation exchange, activation of intestinal enzymes, and alteration of microflora of the gut. Dietary fibers also have great adsorption potency for less polar compounds. Further, dietary fibers have a great binding capacity for various types of bile salts.

Detoxification is a process of cleansing or ridding the body of the many toxins and mutagens that enter the body daily, such as heavy metals, mutagens, toxins, nicotine, dioxins, sodium and cholesterol. This process is important for maintaining overall general health and well being, as a buildup of toxins and mutagens is known to cause certain cancers and other diseases. These toxins and mutagens enter the human body everyday in foods through additives, cooking, and microbial action. Detoxification compositions exist which are unnatural, chemical cleansers. Additionally, herbal detoxification compositions exist which are not effective alone. Accordingly, a detoxification composition has been developed which is natural, and effective when ingested.

### SUMMARY OF THE INVENTION

One object of the present invention is for an ingested detoxification composition having components with synergistic effects.

A more specific object of the present invention is to overcome one or more of the problems discussed above.

Another object of the present invention is for a natural and effective detoxification composition.

Still another object of the present invention is for a detoxification composition having the ability to cleanse the human body of heavy metals, mutagens, toxins, nicotine, dioxins, sodium, and cholesterol.

A further object of the present invention is for a detoxification composition having the ability to reduce cholesterol levels by binding and eliminating bile acids.

A still further object of the present invention is for a detoxification composition which prevents or protects against colon cancer by reducing putrefying bacteria thereby reducing luminal toxins, thickening colonic mucosa, and increasing fecal water content thereby reducing luminal toxins.

In accordance with the present invention, a composition containing alginate for detoxifying and cleansing the human body of toxins and mutagens has been developed. In one form, the detoxification composition can be in the form of a beverage, capsule, pill, lozenge, or other suitable form.

The detoxification composition of the present invention includes alginate, and an ulva rigida extract that has been developed specifically for the purpose of detoxifying the body. The synergistic effects of the components of the detoxification composition of the present invention detoxify and cleanse the human body of toxins and mutagens.

The detoxification composition of the present invention also reduces cholesterol levels, and can protect against colon cancer.

In one embodiment, the detoxification composition can include about 0.01% to 10% (w/v) alginate and about 0.01% to 10% (w/v) ulva rigida extract, in water.

In one embodiment, the detoxification composition can also include N-acetyl-cysteine.

The present invention also relates to a method of detoxifying and cleansing the human body of toxins and mutagens. The method includes administering to a human a detoxification composition comprising alginate, and ulva rigida extract, and the synergistic effects of the components detoxifying and cleansing the body of toxins and mutagens.

These and other embodiments are more fully described in connection with the detailed description.

### DETAILED DESCRIPTION OF THE INVENTION

The present invention relates to a composition containing alginate used for detoxifying and cleansing the human body of toxins and mutagens. The present invention further relates to a composition used to prevent and protect against colon cancer and reduce cholesterol levels in humans. In one form, the composition is designed to be ingested and taken as a beverage.

One embodiment of the present invention relates to a detoxification composition having alginate, and ulva rigida extract.

The synergistic effects of the components of the detoxification composition of the present invention detoxify and cleanse the human body of toxins and mutagens. The detoxification composition of the present invention also reduces cholesterol levels, and prevents or protects against colon cancer. The alginate and ulva rigida extract can work synergistically together by binding to different metals. Specifically, the alginate contains a high content of guluronic acid to bind to metals, mutagens and toxins and a high content of mannuronic acid to stimulate colonic mucin production and to protect the colon from harmful toxins, metals and mutagens through the uronic acid residues. The ulva rigida extract works synergistically with the alginate and contains ulvan which binds to metals and glucuronic acid which binds to toxins making them more water soluble for exiting the body more easily.

Alginate has the ability to form a gel when its various polymer strands are cross-linked with divalent or trivalent cations. Alginate is also believed to have the ability to bind to metals in this manner. The intake of dietary alginates alone results in a number of potentially beneficial physiological effects, such as reduced intestinal absorption, increased satiety, reduced damaging potential GI luminal contents, modulation of colonic microflora, and elevation of colonic barrier function.

Alginate is believed to block the absorption of metal ions, cholesterol and glucose by various methods wherein the levels of total cholesterol, blood glucose, and insulin are reduced. Further, alginates contain guluronic acid and an increase in guluronic acid is believed to increase the affinity of alginates for divalent cations such as Pb²⁺, Cu²⁺, Cd²⁺, Zn²⁺, and Ca²⁺ and makes the metals and toxins more water soluble.

In one embodiment of the present invention, the detoxification composition can include about 0.01% to 10% (w/v) alginate. In a further embodiment of the present invention, the detoxification composition can include about 0.5% to 6% (w/v) alginate. The detoxification composition can still further include about 0.1% to 1.5% (w/v) alginate. In an even further embodiment of the present invention, the detoxification composition can include at least about 0.1% (w/v) alginate.

In one embodiment of the present invention, the detoxification composition can include about 0.05 g to 50 g alginate in 500 mL of water. In a further embodiment of the present invention, the detoxification composition can include about 0.25 g to 30 g alginate in 500 mL of water. The detoxification composition can still further include about 0.5 g to 7.5 g alginate in 500 mL of water. In an even further embodiment of the present invention, the detoxification composition can include at least about 0.5 g alginate in 500 mL of water.

In a further embodiment of the present invention, the detoxification composition can include at least about 0.5 g alginate.

Ulva rigida extract is a species of seaweed and contains ulvan, a soluble polysaccharide fiber. Ulva rigida extract is believed to bind to metals and minerals from acidic solutions, such as cadmium, chromium, copper, zinc, and nickel. It is biosorbent and believed to remove heavy metals from water solutions. Ulva rigida extract also is believed to stimulate mucin production.

In one embodiment of the present invention, the detoxification composition can include about 0.01% to 10% (w/v) ulva rigida extract. In a further embodiment of the present invention, the detoxification composition can include about 0.5% to 6% (w/v) ulva rigida extract. The composition can still further include about 0.1% to 1.5% (w/v) ulva rigida extract. In an even further embodiment of the present invention, the detoxification composition can include at least about 0.1% (w/v) ulva rigida extract.

In one embodiment of the present invention, the detoxification composition can include about 0.05 g to 50 g ulva rigida extract in about 500 mL of water. In a further embodiment of the present invention, the detoxification composition can include about 0.25 g to 30 g ulva rigida extract in about 500 mL of water. The composition can still further include about 0.5 g to 7.5 g ulva rigida extract in about 500 mL of water. In an even further embodiment of the present invention, the detoxification composition can include at least about 0.5 g ulva rigida extract in 500 mL of water.

In a further embodiment of the present invention, the detoxification composition can include at least about 0.5 g ulva rigida extract.

The alginate and ulva rigida extract of the present invention work synergistically to improve the detoxification and cleansing of the human body of heavy metals, mutagens, and toxins, including nicotine, dioxin, sodium, and cholesterol; reduce cholesterol levels; and prevent or protect against colon cancer.

The present invention is believed to absorb food mutagens, and toxins, such as dioxin. The present invention binds to metal ions such as Hg²⁺, and Pb²⁺, and transports the metal ions out of the body. The present invention reduces putrefying bacteria resulting in the reduction in luminal toxins such as sulphide ammonia and phenols. The present invention also binds and eliminates bile acids resulting in lowering cholesterol levels. The present invention further dilutes toxins and lowers colonic luminal toxicity by increasing fecal water content.

In another embodiment, the ratio of alginate, and ulva rigida extract is 5:1

In another embodiment, the ratio of alginate, and ulva rigida extract is 1:5.

In another embodiment, the ratio of alginate, and ulva rigida extract is 1:1.

In another embodiment, the alginate can also be high in guluronic acid to bind metals, mutagens, and toxins. In a further embodiment, the alginate can include 50-100% guluronic acid.

In another embodiment, the alginate can also be high in mannuronic acid to stimulate, and increase colonic mucin production and thicken colonic mucosa. In a further embodiment, the alginate can include 50-100% mannuronic acid.

In another embodiment, the ulva rigida extract can also be high in glucuronic acid which binds to toxic substances making them more water soluble and allowing for elimination. In a further embodiment, the ulva rigida extract can include 50-100% glucuronic acid.

In another embodiment, the alginate can be sodium alginate.

In another embodiment, the detoxification composition can also include N-acetyl-cysteine. N-acetyl-cysteine is a mucolytic agent and has anti-inflammatory effects, even at low dosage levels. N-acetyl-cysteine is an anti-oxidant which scavenges free radicals, and stabilizes the cellular redox status wherein cellular apoptosis, angiogenesis, growth and inflammatory responses are regulated. N-acetyl-cysteine also reduces oxidative stress at both high and low concentrations and in both acute and chronic conditions. N-acetyl-cysteine is believed to chelate heavy metals by binding to the heavy metals such as mercury and lead and removing them from the body. N-acetyl-cysteine also stimulates the production of glutathione, an anti-oxidant which maintains vitamins C and E in their anti-oxidant states and scavenges and removes reactive oxygen species. Glutathione further conjugates fat soluble toxins, such as heavy metals, and converts them to neutral water soluble compounds that can easily be excreted from the body.

In one embodiment of the present invention, the detoxification composition can include about 0.01% to 1% (w/v) N-acetyl-cysteine. In another embodiment of the present invention, the detoxification composition can include about 0.04% to 0.5% (w/v) N-acetyl-cysteine. In a further embodiment of the present invention, the detoxification composition can include about 0.12% to 0.3% (w/v) N-acetyl-cysteine. In an even further embodiment of the present invention, the detoxification composition can include at least about 0.12% (w/v) N-acetyl-cysteine.

In one embodiment of the present invention, the detoxification composition can include about 50 mg to 5 g N-acetyl-cysteine in 500 mL of water. In another embodiment of the present invention, the detoxification composition can include about 200 mg to 2.5 g N-acetyl-cysteine in 500 mL of water. In a further embodiment of the present invention, the detoxification composition can include about 600 mg to 1.5 g N-acetyl-cysteine in 500 mL of water. In an even further embodiment of the present invention, the detoxification composition can include at least about 600 mg N-acetyl-cysteine in 500 mL of water.

In an even further embodiment of the present invention, the detoxification composition can include at least about 600 mg N-acetyl-cysteine.

In another embodiment, the detoxification composition can also include dandelion extract or burdock extract to further detoxify and cleanse the human body of toxins and mutagens.

In another embodiment, the detoxification composition can include a flavor component, a sweetener component and/or combinations thereof for improved taste.

In one embodiment, the detoxification composition can include about 0.01% to 10% (w/v) alginate and about 0.01% to 10% (w/v) ulva rigida extract. In another embodiment, the detoxification composition can include about 0.01% to 10% (w/v) alginate, about 0.01% to 10% (w/v) ulva rigida extract, and about 0.01% to 1% (w/v) N-acetyl-cysteine.

The following example of one embodiment of the present invention provides a detoxification composition in about 500 ml of water and demonstrates the scope of the present invention. The detoxification composition includes:

about 0.1% (w/v) alginate;

about 0.1% (w/v) ulva rigida; and

1 to 3 drops fruit concentrate.

The following example of one embodiment of the present invention provides a detoxification composition in about 500 ml of water and demonstrates the scope of the present invention. The detoxification composition includes:

about 0.1% (w/v) alginate;

about 0.1% (w/v) ulva rigida;

about 0.12% (w/v) N-acetyl cysteine; and

1 to 3 drops fruit concentrate.

Another example of one embodiment of the present invention provides a detoxification composition in about 500 ml of water and demonstrates the scope of the present invention. The detoxification composition includes:

about 0.5 g alginate;

about 0.5 g ulva rigida; and

about 1 drop of fruit concentrate.

Another example of one embodiment of the present invention provides a detoxification composition in about 500 ml of water and demonstrates the scope of the present invention. The detoxification composition includes:

about 0.5 g alginate;

about 0.5 g ulva rigida;

about 600 mg N-acetyl-cysteine; and

about 1 drop of fruit concentrate.

A further example of one embodiment of the present invention provides a detoxification composition in water and demonstrates the scope of the present invention. The detoxification composition includes:

about 0.5 g alginate; and

about 0.5 g ulva rigida extract.

A further example of one embodiment of the present invention provides a detoxification composition in water and demonstrates the scope of the present invention. The detoxification composition includes:

about 0.5 g alginate;

about 0.5 g ulva rigida extract; and

about 600 mg N-acetyl-cysteine.

In another embodiment, the detoxification composition can be further sterilized and have an extended shelf-life through an ultra-high temperature process. The ultra-high temperature process includes heating the detoxification composition for about 2-3 seconds at a temperature of at least approximately 275 degrees Fahrenheit. The ultra-high temperature process aids in killing microbes present in the detoxification composition prior to mixing in the water.

In one embodiment of the present invention, the detoxification composition can be a beverage. Suitably, the detoxification composition can have a flavor and can be consumed as a snack. In another embodiment of the present invention, the detoxification composition can be in the form of a pill, a capsule, a powder, a chewable, or syrup. In another embodiment, the detoxification composition can be in a cereal bar that does not contain calcium or acid for flavoring.

In another embodiment, the detoxification composition can include a preservative. The preservative can include parabens and can further include methyl and propyl parabens. Parabens are common preservatives in the cosmetic, food and beverage, and pharmaceutical industries due to their anti-microbial effects. In the U.S., parabens are GRAS approved and allowed at a maximum concentration of 0.1%. In a further embodiment, the detoxification composition can include methyl and propyl parabens in a 3:1 or 2:1 ratio. In a further embodiment, the detoxification composition can include 0.375 g of methyl paraben and 0.125 g of propyl paraben.

The present invention also relates to a method of detoxifying and cleansing the human body of mutagens and toxins. The method includes administering a detoxification composition comprising alginate, and ulva rigida extract, and the synergistic effects of the components detoxifying and cleansing the body of metals, toxins and mutagens. The method can include binding of multivalent metal ions such as Hg²⁺ and Pb²⁺, and transporting the metal ions out of the body. The method can include absorbing food mutagens and toxins, such as dioxin.

The method further includes preventing and protecting against colon cancer by reducing putrefying bacteria resulting in the reduction in luminal toxins such as ammonia and phenols.

The method further includes thickening the colonic mucosa because the high mannuronic acid in the alginate of the detoxification composition has the ability to stimulate colonic mucin secretion. Mucins are involved in cytoprotective functions against bacteria, toxins, luminal proteases, and carcinogens.

The method further includes alginate binding and eliminating bile acids and salts, which are synthesized in the liver from cholesterol, resulting in the reduction of cholesterol levels. The alginate has an ability to inhibit absorption in the gut and eliminate bile salts from the body. The alginate further disperses in water retaining cholesterol, and inhibiting lipid absorption in the gastrointestinal tract.

The method further includes the ulva rigida extract containing glucuronic acid of the detoxification composition binding toxins, making them more water soluble, and eliminating the toxins. The ulva rigida extract further dilutes the toxins and lowers colonic luminal toxicity by increasing fecal water content.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A detoxification composition, comprising:
alginate; and
ulva rigida extract.

2. The composition of claim 1 wherein the composition is in a beverage form.

3. The composition of claim 1 wherein the composition is in a powder, capsule, tablet, chewable or syrup.

4. The composition of claim 1 wherein the alginate comprises sodium alginate.

5. The composition of claim 1 wherein the ratio of alginate to ulva rigida extract is between 5:1 and 1:5.

6. The composition of claim 1 wherein the ratio of alginate to ulva rigida extract is 1:1.

7. The composition of claim 1 further comprising dandelion extract and burdock extract.

8. The composition of claim 1 wherein the alginate comprises about 50-100% of guluronic acid.

9. The composition of claim 1 wherein the alginate comprises about 50-100% of mannuronic acid.

11. The composition of claim 1 wherein the ulva rigida extract comprises ulvan.

12. The composition of claim 11 wherein the ulvan comprises 50-100% of glucuronic acid.

13. The composition of claim 1 further comprising a flavoring component, a sweetening component and combinations thereof.

14. The composition of claim 1 wherein the alginate is about 0.01% to 10% (w/v) and the ulva rigida extract is about 0.01% to 10% (w/v), in water.

15. The composition of claim 1 wherein the alginate is about 0.05 g to 50 g and the ulva rigida extract is about 0.5 g to 50 g, in water.

16. The composition of claim 1 wherein the alginate is about 0.5 g and the ulva rigida extract is about 0.5 g, in water.

17. The composition of claim 1 further comprising N-acetyl-cysteine.

18. The composition of claim 17 wherein the N-acetyl-cysteine is about 0.01% to 1% (w/v), in water.

19. The composition in claim 17 wherein the N-acetyl-cysteine is about 50 mg to 5 g, in water.

20. The composition in claim 17 wherein the N-acetyl-cysteine is about 600 mg, in water.

21. A detoxification composition, comprising:
at least about 0.1% (w/v) alginate; and
at least about 0.1% (w/v) ulva rigida extract,
in about 500 mL of water.

22. The composition of claim 21 further comprising about 1 to 3 drops of fruit concentrate.

23. The composition of claim 21 further comprising:
at least about 0.12% (w/v) N-acetyl cysteine.

24. A method for detoxifying and cleansing the human body comprising the steps of:
administering to a human the detoxification composition in accordance with claim 1.

25. A method for reducing cholesterol comprising the steps of:
administering to a human the detoxification composition in accordance with claim 1.
